(19) United States
(12) Patent Application Publication
(10) Pub. No.: US 2016/0176776 A1
(43) Pub. Date: Jun. 23, 2016

(54) CONVERSION OF OXYGENATES TO AROMATICS

(71) Applicants: Samia Ilias, Somerville, NJ (US); Brett Loveless, Houston, TX (US); Stephen J. McCarthy, Center Valley, PA (US); Rohit Vijay, Bridgewater, NJ (US)

(72) Inventors: Samia Ilias, Somerville, NJ (US); Brett Loveless, Houston, TX (US); Stephen J. McCarthy, Center Valley, PA (US); Rohit Vijay, Bridgewater, NJ (US)

(73) Assignee: ExxonMobil Research and Engineering Company, Annandale, NJ (US)

(21) Appl. No.: 14/956,446

(22) Filed: Dec. 2, 2015

Related U.S. Application Data
(60) Provisional application No. 62/095,191, filed on Dec. 22, 2014.

(51) Int. Cl. C07C 1/20; B01J 29/40

(52) U.S. Cl. CPC C07C 1/20; B01J 29/405; C07C 2529/40

(57) ABSTRACT

Described herein are processes for production of hydrocarbon products comprising contacting a feed comprising methanol and/or dimethyl ether with a catalyst composition, which comprises a zeolite having a constraint index from 1-12 and an active binder comprising a metal oxide with a dehydrogenation function, under conditions sufficient to form the hydrocarbon product, wherein the hydrocarbon product comprises aromatics, olefins, and/or paraffins. Also described herein are catalyst compositions comprising a zeolite having a 10-/12-membered ring framework and a microporous surface area of at least 150 m²/g, and from ˜1 wt % to ˜10 wt % of a zinc oxide binder, the catalyst composition having a zinc to aluminum atomic ratio from ˜0.08 to ˜8.5.

### CROSS-REFERENCE TO RELATED APPLICATIONS

This application claims the benefit of provisional U.S. Ser. No. 62/095,191, filed Dec. 22, 2014, the entire contents of which are expressly incorporated by reference herein.

### FIELD OF THE INVENTION

This invention relates to a process for converting oxygenates to aromatic hydrocarbons.

### BACKGROUND

Benzene, toluene, and xylenes (BTX) are basic building blocks of the modern petrochemical industry. The present source of these compounds primarily is the refining of petroleum. As petroleum supplies dwindle, so does the supply of benzene, toluene, and xylenes. Thus, there is a need to develop alternative sources for these compounds.

Development of fossil fuel conversion processes has enabled the production of oxygenated hydrocarbons from coal, natural gas, shale oil, etc. Synthesis gas (containing at least CO and H₂) is readily obtained from the fossil fuels and can be further converted to lower aliphatic oxygenates, especially methanol (MeOH) and/or dimethyl ether (DME). U.S. Pat. No. 4,237,063 discloses the conversion of synthesis gas to oxygenated hydrocarbons using metal cyanide complexes. U.S. Pat. No. 4,011,275 discloses the conversion of synthesis gas to methanol and dimethyl ether by passing the mixture over a zinc-chromium acid or copper-zinc-alumina acid catalyst. U.S. Pat. No. 4,076,761 discloses a process for making hydrocarbons from synthesis gas wherein an intermediate product formed is a mixture of methanol and dimethyl ether.

Methanol to gasoline (MTG) is a commercial process in which methanol is converted over an H-ZSM-5 catalyst to gasoline boiling range hydrocarbon products. MTG processes are, for example, described in U.S. Pat. No. 3,894,106. In the MTG process, methanol is first dehydrated to form dimethyl ether, which is then converted to olefins. The olefins undergo further reactions, including bimolecular hydrogen transfer and cyclization, eventually resulting in the production of three paraffins for every one aromatic. The resulting product distribution of a MTG process is a high quality gasoline composed primarily of aromatics and paraffins.

The addition of transition metals to an MTG catalyst provides an alternative pathway to olefin dehydrogenation by promoting the formation of molecular H₂. Thus, the addition of a transition metal to H-ZSM-5 catalysts allows for aromatic formation without the concurrent formation of paraffins. Typically, transition metals are added to H-ZSM-5 via metal impregnation by incipient wetness or creating an intraparticle mixture of the metal (as the zero-valent metal or as a metal oxide or in a cationic state) with H-ZSM-5.

There remains, however, a continuing need to increase the yield of aromatics and olefins, as compared to paraffins, in the conversion of oxygenates to hydrocarbons over zeolite catalysts, such as ZSM-5.

### SUMMARY

According to the present invention, it has now been found that a significant increase in aromatic and olefin yields in the conversion of methanol and/or dimethyl ether over a bound zeolite catalyst can be achieved by employing an active binder comprising a metal oxide with a dehydrogenation function.

Thus, in one aspect, the invention relates to a process for production of a hydrocarbon product comprising contacting a feed comprising methanol and/or dimethyl ether with a catalyst composition, which comprises a zeolite having a constraint index from 1 to 12 and an active binder comprising a metal oxide with a dehydrogenation function (which can optionally comprise or be one or more of Ga₂O₃, CrOₓ, and ZnO), under conditions sufficient to form the hydrocarbon product, wherein the hydrocarbon product comprises one or more of aromatics, olefins, and paraffins.

In another aspect, the invention relates to a catalyst composition comprising:

a zeolite having a 10-membered or 12-membered ring framework and a microporous surface area of at least 150 m²/g; and an active binder comprising zinc oxide in an amount from 1 wt % to 10 wt % of the catalyst composition, the catalyst composition having a zinc to aluminum atomic ratio from 0.08 to 8.5.

### BRIEF DESCRIPTION OF THE DRAWINGS

FIG. 1 shows the aromatic yield (wt % of hydrocarbon products) for H-ZSM-5 catalysts bound with ˜0-35 wt % ZnO during methanol conversion. The horizontal axis represents wt % ZnO binder in the catalyst; the vertical axis represents wt % aromatics in the hydrocarbon product.

### DETAILED DESCRIPTION OF THE EMBODIMENTS

The present invention uses a reactive metal oxide binder having a dehydrogenation function in the preparation of the MTG catalyst and can advantageously show a significant and unexpected increase in yields of aromatics (or in yields of unsaturated compounds generally, such as aromatics plus olefins) compared to a typical MTG catalyst. In some embodiments of the invention, the yield of unsaturates (e.g., aromatics and/or olefins) can be at least 40% of the hydrocarbons in the product, for example at least 60 wt %, at least 70 wt %, or at least 80%; additionally or alternately, the yield of unsaturates (e.g., aromatics and/or olefins) can be 99 wt % or less of the hydrocarbons in the product, for example 98 wt % or less, 97 wt % or less, 95 wt % or less, 90 wt % or less, or 80 wt % or less.

Use of the catalyst composition of the invention in a MTG process can advantageously allow capture of hydrogen gas as a valuable product from the reaction. Also, in some embodiments of the invention, the amount of paraffins in the product can be advantageously low, such as less than 40 wt % of the hydrocarbons in the product, for example less than 30 wt %.

In embodiments of the invention, a metal oxide having a hydrogenation function, such as including one or more of Ga₂O₃, CrOₓ and ZnO, particularly including or being ZnO, can be added to the catalyst composition in an amount from about 0.5 wt % to about 20 wt %, based on the final weight of the catalyst composition.

An “active binder” for purposes of this invention is a binder material comprising a metal oxide that imparts a hydrogenation function to the binder. Thus, in the present invention, the metal oxide having a hydrogenation function can be added to the catalyst composition as an active binder. Use of the catalyst composition of the invention in an MTG process can unexpectedly provide a hydrocarbon product containing an increased proportion of unsaturates (e.g., aromatics plus olefins) and/or a decreased proportion of paraffins, compared to state of the art MTG processes.

The catalyst composition of the invention can include a zeolite having a Constraint Index from 1 to 12 (as defined in U.S. Pat. No. 4,016,218) and can include an active binder comprising a metal oxide, particularly comprising zinc oxide (ZnO).

Suitable zeolites can include, but are not necessarily limited to, ZSM-5, ZSM-11, ZSM-12, ZSM-22, ZSM-23, ZSM-35, ZSM-48, and the like, as well as combinations thereof. ZSM-5 is described in detail in U.S. Pat. No. 3,702,886 and RE 29,948. ZSM-11 is described in detail in U.S. Pat. No. 3,709,979. ZSM-12 is described in U.S. Pat. No. 3,832,449. ZSM-22 is described in U.S. Pat. No. 4,556,477. ZSM-23 is described in U.S. Pat. No. 4,076,842. ZSM-35 is described in U.S. Pat. No. 4,016,245. ZSM-48 is more particularly described in U.S. Pat. No. 4,234,231. In certain embodiments, the zeolite can comprise, consist essentially of, or be ZSM-5, advantageously in its acid or phosphate/acid form.

The zeolite employed in the present catalyst composition, particularly when it has an MEL and/or MFI framework type, can typically have a silica to alumina molar ratio of at least 20, e.g., at least 40, at least 60, from about 20 to about 200, from about 20 to about 100, from about 20 to about 80, from about 40 to about 200, from about 40 to about 100, or from about 40 to about 80.

When used in the present catalyst composition, the zeolite can advantageously be present at least partly in the hydrogen form. Depending on the conditions used to synthesize the zeolite, this may implicate converting the zeolite from, for example, the alkali (e.g., sodium) form. This can readily be achieved, e.g., by ion exchange, to convert the zeolite to the ammonium form, followed by calcination in air or an inert atmosphere at a temperature from about 400° C. to about 700° C. to convert the ammonium form to the active hydrogen form. If an organic structure directing agent is used in the synthesis of the zeolite, additional heat treatments/calcinations, or different conditions for calcinations, may be desirable to at least partially remove/decompose the organic structure directing agent.

To enhance the steam stability of the zeolite without excessive loss of its initial acid activity, the present catalyst composition can contain, and/or can be treated to contain, phosphorus in an amount between about 0.01 wt % and about 3 wt % on an elemental phosphorus basis, e.g., between about 0.05 wt % and about 2 wt %, based on the total catalyst composition. The phosphorus can be added to the catalyst composition at any stage during synthesis of the zeolite and/or formulation of the zeolite and binder into the catalyst composition. Generally, phosphorus addition for steam stability can be achieved by treatment, e.g., by spraying and/or by impregnating an almost-final catalyst composition (and/or a precursor thereto, typically at least after zeolite formation) with a solution of a phosphorus compound. Suitable phosphorus compounds can include, but need not be limited to, phosphinic acid [H₂PO(OH)], phosphonic acid [HPO(OH)₂], phosphoric [PO(OH)₃] acid, salts thereof, esters thereof, phosphorus halides, and the like, and combinations thereof. After any phosphorus treatment(s), the catalyst can generally be calcined, e.g., in air, at a temperature from about 400° C. to about 700° C. to at least partially (or particularly to substantially) convert/decompose the organic portion of the phosphorus compound into a phosphorus oxide form.

The bound, and particularly also phosphorus-stabilized, zeolite catalyst composition employed herein can be characterized by at least one, at least two, or all of the following properties: (a) a microporous surface area of at least 150 m²/g, advantageously at least 340 m²/g or at least 375 m²/g; (b) a diffusivity for 2,2-dimethylbutane of greater than 1.2×10⁻² sec⁻¹, when measured at a temperature of about 120° C. and a 2,2-dimethylbutane pressure of about 60 torr (about 8 kPa); (c) an alpha value after steaming in about 100% steam for about 96 hours at about 1000° F. (about 538° C.) of at least 20, e.g., at least 40; (d) mesopore size distribution with less than 20% of mesopores having a size below 10 nm; and (e) a mesopore size distribution with more than 60% of mesopores having a size at least 21 nm after steaming in approximately 100% steam for about 96 hours at about 1000° F. (about 538° C.). It should be appreciated by one of ordinary skill in the art that properties (a), (b), and (d) above, unlike properties (c) and (e), are measured before any steaming of the catalyst composition.

Of these properties, microporosity and diffusivity for 2,2-dimethylbutane can be determined by a number of factors, including but not necessarily limited to the pore size and crystal size of the zeolite and the availability of the zeolite pores at the surfaces of the catalyst particles. Mesopore size distribution can be determined mainly by surface area measurements of the bound form. Given the disclosure herein regarding the use of a relatively low surface area binder, producing a zeolite catalyst with the desired mesopore size distribution, microporous surface area, and 2,2-dimethylbutane diffusivity should be well within the expertise of anyone of ordinary skill in the zeolite chemistry art.

### Alpha Value

MTG reactions are typically catalyzed over acid sites. The acidity of the catalyst can tend to decrease with time on stream in the MTG reactor. In order to assess the ability of the catalyst to withstand hydrothermal stress in the MTG reactor, the steaming conditions in the MTG reactor can be simulated by a hydrothermal treatment in a laboratory reactor. The acidity of the catalysts can then be measured by their n-hexane cracking activity (alpha test).

The n-hexane cracking activity, expressed as “alpha value”, can be a measure for the acidity of the catalyst. Alpha value is defined as the ratio of the first order rate constant for n-hexane cracking, relative to a silica-alumina standard, and can be determined using the following formula:

alpha=A*In(1−X)/π

where A includes the reference rate constant and unit conversion, about −1.043; where X represents the fractional conversion; and where π represents residence time and equals wt*(ρ*F), with ρ being the packing density (in g/cm³), F being the gas flow rate (in cm³/min), and “wt” being the catalyst weight (in grams).

Alpha value can be a useful measure of the acid activity of a zeolite catalyst, as compared with a standard silica-alumina catalyst. The alpha test is described in U.S. Pat. No. 3,354,078; in the Journal of Catalysis, v. 4, p. 527 (1965); v. 6, p. 278 (1966); and v. 61, p. 395 (1980), each incorporated herein by reference as to that description. The experimental conditions of the test can include a constant temperature of about 538° C. and a variable flow rate, as described in detail in the Journal of Catalysis, v. 61, p. 395. Higher alpha values can generally correspond to a more active cracking catalyst. Since the present catalyst composition may be used in reactions such as MTG, where the zeolite might be subject to hydrothermal degradation (e.g., dealumination) of the zeolite, it can be important for the catalyst composition to retain a significant alpha value, for example at least 20, after steaming in about 100% steam for about 96 hours at about 1000° F. (about 538° F.).

### Diffusivity for 2,2-Dimethylbutane:

The porosity of a zeolite can play a role in product selectivity and/or coke formation in reactions involving the zeolite. Fast diffusion of reactants into and of products out of zeolite micropores can be desirable to obtain the desired product composition and/or to prevent coke formation. Diffusivity of 2,2-dimethylbutane (2,2-DMB) can be calculated from the rate of 2,2-DMB uptake and the amount of hexane uptake using the following formula:

D/r²=k*(2,2-DMB uptake rate/hexane uptake)

where D/r² is the diffusivity [10⁻⁶ sec⁻¹], where 2,2-DMB uptake rate is in units of mg/g/min^{0.5}, where hexane uptake is in units of mg per g of catalyst, and where k is a proportionality constant.

Hexane and 2,2-DMB uptakes can be measured in two separate experiments using a microbalance. Prior to hydrocarbon adsorption, about 50 mg of the catalyst sample can be heated in air for about 30 minutes to about 500° C., in order to remove moisture and hydrocarbon/coke impurities. For hexane adsorption, the sample can be cooled to about 90° C. and subsequently exposed to a flow of about 100 mbar (about 10 kPa) of hexane in nitrogen at about 90° C. for about 40 minutes. For 2,2-DMB adsorption, the catalyst sample can be cooled to about 120° C. after the air calcination step and exposed to 2,2-dimethylbutane at a pressure of about 60 torr (about 8 kPa) for about 30 minutes. The formulation of a zeolite into an extrudate with a binder can result in blockage and/or narrowing of pore openings in the zeolite. For zeolite structures with otherwise equivalent framework types/pore sizes, a higher 2,2 DMB diffusivity can suggest a larger degree of unobstructed zeolite channels and pore openings.

A particular zeolite for use in the invention can include or be ZSM-5. The zeolite can advantageously be provided in its acid form, e.g. H-ZSM-5, or in its acidic, phosphorus modified form, e.g. Ph/H-ZSM-5.

The catalyst composition of the invention can advantageously include the zeolite in the form of small crystals, e.g., having an average size of less than or equal to 0.5 microns, for example less than 0.3 microns or less than 0.1 microns. Such small crystals of ZSM-5 can be particularly advantageous for use in the process of the invention.

The catalyst composition of the invention can optionally comprise an inactive binder or other porous matrix material distinct from the “active binder”, for example silica, titania, various natural clays, or the like. The inactive binder can typically comprise or be alumina, silica, or silica-alumina, which can be selected so as to have a surface area less than 200 m²/g, for example less than 150 m²/g or less than or equal to 100 m²/g. Suitable examples of inactive alumina binders can comprise or be Pural™ 200 and/or Versal™ 300 alumina. When an inactive binder and/or other porous material is used, the binder or porous material can be present in an amount from about 1 wt % to about 60 wt % (e.g., between about 1 wt % and about 50 wt % or between about 5 wt % and about 40 wt %), based on the weight of the catalyst composition overall.

The catalyst composition of the invention can advantageously include an active binder in an amount from ˜0.5 wt % to ˜15 wt %, for example from ˜0.5 wt % to ˜10 wt %, from ˜1.0 wt % to ˜15 wt %, from ˜1.0 wt % to ˜10 wt %, from ˜1.3 wt % to ˜15 wt %, or from ˜1.3 wt % to ˜10 wt %, based on the weight of the composition.

When zinc oxide is present in the metal oxide active binder, the amount of active binder added can function to provide zinc in an amount of ˜0.05 wt % to ˜10 wt %, for example from ˜0.8 wt % to ˜6 wt %, based on the weight of the catalyst composition overall. Thus, the catalyst composition of the invention can advantageously have a zinc to aluminum atomic ratio from ˜0.08 to ˜8.5, for example from ˜0.1 to ˜4.5.

In a particular embodiment of the invention, the zeolite can be characterized by a 10-12-membered ring framework, a microporous surface area of at least 150 m²/g, and a silica to alumina molar ratio from about 20 to about 100. In this particular embodiment, the zeolite may further have a constraint index from 1 to 12, may comprise or be ZSM-5, and can preferably be in an acid form.

The preparation of a zeolite in a phosphorus-modified form that can also have acidic sites is described, for example, in U.S. Patent Application Publication No. 2013/0102825, which is hereby incorporated by reference in its entirety and for all purposes, though it is particularly useful for its disclosure regarding phosphorus-modified acidic forms of zeolites.

In a particular embodiment, a catalyst composition according to the invention can be prepared by adding an active binder comprising zinc oxide (ZnO) in an amount of ˜1 wt % to ˜10 wt %, based on the weight of the catalyst composition, such that the final catalyst composition can have a zinc to aluminum atomic ratio from ˜0.08 to ˜8.5.

In some embodiments of the catalyst according to the invention, substantially all of the zinc present in the catalyst composition (particularly substantially all of the zinc intentionally added, e.g., not including zinc contaminants in the reactants/components) can be present in the active binder.

Additional binder and/or porous matrix materials can optionally be added to the catalyst composition in any of the typical ways of adding a binder to a zeolite catalyst composition; generally the binder material can be mixed together with the zeolite and then extruded/further processed, e.g., to provide catalyst material having desired particle size and/or other physical/chemical properties. See, for example, U.S. Pat. No. 3,760,024, hereby incorporated by reference in its entirety.

For instance, a mixture of synthesized zeolite, perhaps containing an organic directing agent used in its synthesis, can be blended in a muller with the desired amount of the binder. The binder can include the active binder and can optionally include a desired amount of inactive binder and/or a desired amount of one or more porous matrix materials. The blend can then be extruded, and the resultant extrudate can be calcined. This calcining can be performed in a non-oxidizing atmosphere, for example nitrogen, and for a desired time, for instance for about 3 hours, and at a desired temperature, for example about 1000° F. (about 538° C.). If an organic directing agent has been used in the synthesis, the calcining conditions should be sufficient to at least partially (and in most cases substantially) decompose into carbonaceous deposits and/or remove, e.g., as various gaseous carbonaceous oxide products, any organic template that might be present.

In certain embodiments, the calcined extrudate can then be exchanged with an ammonium nitrate solution to convert the zeolite from an alkali (e.g., sodium) to an ammonium form, whereafter the extrudate can again be calcined in air under conditions sufficient to convert the zeolite from an ammonium to an active (e.g., the hydrogen) form, and at the same time sufficient to decompose/remove any remaining trace of the organic directing template by oxidation, for instance for about 3 hours at about 1000° F. (about 538° C.). The thus obtained extrudate can then be impregnated with phosphoric acid to a target level, for example about 1 wt % phosphorus via aqueous incipient wetness impregnation. The sample can then be dried and then yet again calcined in air, for instance for about 3 hours at about 1000° F. (about 538° C.).

In a process according to the invention a feedstock comprising methanol and dialkyl ethers, including or being dimethyl ether, can be contacted with a catalyst composition according to the invention at a temperature from ˜300° C. to ˜600° C., for example from ˜400° C. to ˜550° C. The reaction can advantageously be run at a pressure from about 50 kPaa to about 5000 kPaa, for example from about 100 kPaa to about 1040 kPaa.

### Additional Embodiments

The instant invention can further include one or more of the following embodiments.

### Embodiment 1

A process for production of a hydrocarbon product comprising contacting a feed comprising methanol and/or dimethyl ether with a catalyst composition, which comprises a zeolite having a constraint index from 1 to 12 and an active binder comprising a metal oxide with a dehydrogenation function (which can optionally comprise or be one or more of Ga₂O₃, CrOₓ, and ZnO), under conditions sufficient to form the hydrocarbon product, wherein the hydrocarbon product comprises one or more of aromatics, olefins, and paraffins.

### Embodiment 2

The process according to embodiment 1, wherein the contacting is performed at a temperature from about 300° C. to about 600° C. (e.g., from about 400° C. to about 550° C.) and/or at a pressure from about 50 kPaa to about 5000 kPaa (e.g., from about 100 kPaa to about 1040 kPaa).

### Embodiment 3

The process according to embodiment 1 or embodiment 2, wherein the zeolite comprises an MEL or MFI framework type.

### Embodiment 4

The process according to any one of the previous embodiments, the catalyst composition is characterized by one or more of the following: a silica to alumina molar ratio of the zeolite from about 20 to about 100 (e.g., from about 40 to about 80); a Zn content from about 0.05 wt % to about 10 wt %, based on the weight of the catalyst composition (e.g., from about 0.8 wt % to about 6 wt %); an active binder content from about 0.5 wt % to about 60 wt %, based on the weight of the catalyst composition (e.g., from about 1 wt % to about 10 wt %); a zeolite microporous surface area of at least 150 m²/g; and a zinc to aluminum atomic ratio of about 0.08 to about 8.5.

### Embodiment 5

The process according to any one of the previous embodiments, wherein the zeolite comprises or is a ZSM-5 zeolite, such as H-ZSM-5.

### Embodiment 6

The process according to embodiment 5, wherein the ZSM-5 has an average crystal size less than or equal to 0.5 microns (e.g., less than or equal to 0.1 microns).

### Embodiment 7

The process according to any one of the previous embodiments, wherein the catalyst further comprises phosphorus.

### Embodiment 8

The process according to any one of the previous embodiments, wherein any zinc in the catalyst, other than zinc that might be provided by any contaminants, is present only in the active binder.

### Embodiment 9

The process according to any one of the previous embodiments, wherein the hydrocarbon product has a content of aromatics and olefins of at least 60 wt % (e.g., at least 70 wt %) of hydrocarbons in the product and/or a content of paraffins of less than 40 wt % of hydrocarbons in the product.

### Embodiment 10

A catalyst composition comprising: a zeolite having a 10-membered or 12-membered ring framework and a microporous surface area of at least 150 m²/g; and an active binder comprising zinc oxide in an amount from about 1 wt % to about 10 wt % of the catalyst composition, the catalyst composition having a zinc to aluminum atomic ratio from about 0.08 to about 8.5.

### Embodiment 11

The catalyst composition according to embodiment 10, wherein the catalyst composition is characterized by one or more of the following: a silica to alumina molar ratio of the zeolite from about 20 to about 100 (e.g., from about 40 to about 80); a Zn content from about 0.05 wt % to about 10 wt %, based on the weight of the catalyst composition (e.g., from about 0.8 wt % to about 6 wt %); an active binder content from about 0.5 wt % to about 60 wt %, based on the weight of the catalyst composition (e.g., from about 1 wt % to about 10 wt %); a zeolite microporous surface area of at least 150 m²/g; and a zinc to aluminum atomic ratio of about 0.08 to about 8.5.

### Embodiment 12

The catalyst composition according to embodiment 10 or embodiment 11, wherein the zeolite comprises or is a ZSM-5 zeolite, such as H-ZSM-5.

### Embodiment 13

The catalyst composition according to embodiment 12, wherein the ZSM-5 has an average crystal size less than or equal to 0.5 microns (e.g., less than or equal to 0.1 microns).

### Embodiment 14

The catalyst composition according to any one of embodiments 10-13, wherein the catalyst further comprises phosphorus.

### Embodiment 15

The catalyst composition according to any one of embodiments 10-14, wherein any zinc in the catalyst, other than zinc that might be provided by any contaminants, is present only in the active binder.

### EXAMPLES

The invention can be more particularly described with reference to the following non-limiting Example.

### Example 1

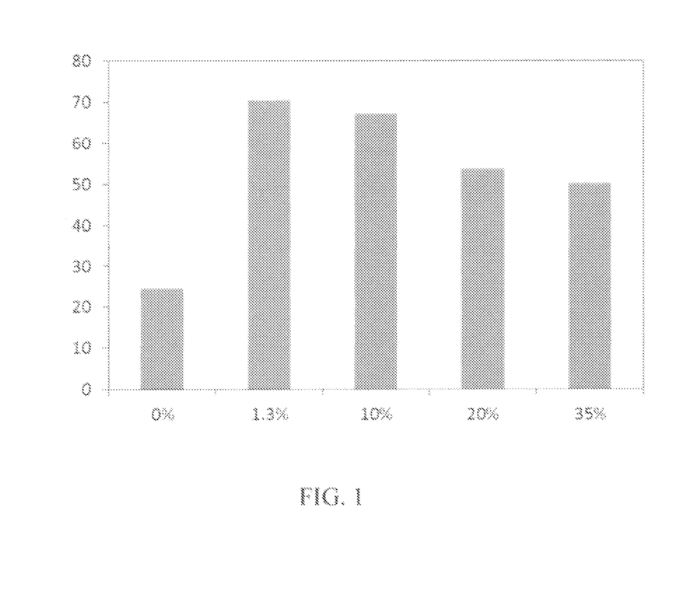

FIG. 1 shows the aromatic yield (wt % of hydrocarbon products) for H-ZSM-5 catalysts bound with 0 wt % to about 35 wt % ZnO during methanol conversion. The reactions were run at ˜500° C., ˜103 kPag (˜1 barg), and ˜20 hr⁻¹ WHSV, so as to attain ˜100% CH₃OH conversion. The “hydrocarbon product” described in FIG. 1 does not include any COₓ or H₂ that may have been generated.

All the catalysts bound with ZnO appeared to show at least a two-fold increase in aromatic yield compared to the H-ZSM-5 catalyst containing 0 wt % ZnO binder. The highest aromatic yields were achieved by converting methanol over H-ZSM-5 catalysts bound with ˜1 wt % to ˜10 wt % ZnO. H-ZSM-5 catalysts bound with more than ˜10 wt % ZnO appeared to show decreases in aromatic yield during methanol conversion.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. A process for production of a hydrocarbon product comprising contacting a feed comprising methanol and/or dimethyl ether with a catalyst composition, which comprises a zeolite having a constraint index from 1-12 and an active binder comprising a metal oxide with a dehydrogenation function, under conditions sufficient to form the hydrocarbon product, wherein the hydrocarbon product comprises one or more of aromatics, olefins, and paraffins.

2. The process of claim 1, wherein the metal oxide comprises one or more of Ga₂O₃, CrOₓ, and ZnO.

3. The process of claim 1, wherein the contacting is performed at a temperature from about 300° C. to about 600° C.

4. The process of claim 1, wherein the contacting is performed at a temperature from about 400° C. to about 550° C.

5. The process of claim 1, wherein the contacting is performed at a pressure from about 50 kPaa to about 5000 kPaa.

6. The process of claim 1, wherein the contacting is performed at a pressure from about 100 kPaa to about 1040 kPaa.

7. The process of claim 1, wherein the zeolite comprises an MEL or MFI framework type.

8. The process of claim 1, wherein the zeolite has a silica to alumina molar ratio from about 20 to about 100.

9. The process of claim 1, wherein the zeolite has a silica to alumina molar ratio from about 40 to about 80.

10. The process of claim 1, wherein the catalyst composition comprises Zn in an amount from about 0.05 wt % to about 10 wt % of the catalyst composition.

11. The process of claim 1, wherein the catalyst composition comprises Zn in an amount from about 0.8 wt % to about 6 wt % of the catalyst composition.

12. The process of claim 1, wherein the catalyst composition comprises the active binder in an amount from about 0.5 wt % to about 60 wt % of the catalyst composition.

13. The process of claim 1, wherein the catalyst composition comprises the active binder in an amount from about 1 wt % to about 10 wt % of the catalyst composition.

14. The process of claim 1, wherein the zeolite has a microporous surface area of at least 150 m²/g.

15. The process of claim 1, wherein the catalyst has a zinc to aluminum atomic ratio from about 0.08 to about 8.5.

16. The process of claim 1, wherein the zeolite comprises ZSM-5.

17. The process of claim 1, wherein the zeolite is H-ZSM-5.

18. The process of claim 16, wherein the ZSM-5 has an average crystal size less than or equal to 0.5 microns.

19. The process of claim 16, wherein the ZSM-5 has an average crystal size less than or equal to 0.1 microns.

20. The process of claim 1, wherein the catalyst further comprises phosphorus.

21. The process of claim 12, wherein substantially all the zinc in the catalyst is present in the active binder.

22. The process of claim 1, wherein the hydrocarbon product has a content of aromatics and olefins that comprises at least 60 wt % of hydrocarbons in the product.

23. The process of claim 1, wherein the hydrocarbon product has a content of aromatics and olefins that comprises at least 70 wt % of hydrocarbons in the product.

24. The process of claim 1, wherein the hydrocarbon product has a content of paraffins that comprises less than 40 wt % of hydrocarbons in the product.

25. A catalyst composition comprising:
a zeolite having a 10-membered or 12-membered ring framework and a microporous surface area of at least 150 m²/g; and
an active binder comprising zinc oxide in an amount from about 1 wt % to about 10 wt % of the catalyst composition,
the catalyst composition having a zinc to aluminum atomic ratio from about 0.08 to about 8.5.

26. The catalyst composition of claim 25, wherein the zeolite has a silica to alumina molar ratio from about 20 to about 100.

27. The catalyst composition of claim 25, wherein the zeolite comprises ZSM-5.

28. The catalyst composition of claim 25, wherein the catalyst further comprises phosphorus.

29. The catalyst composition of claim 25, wherein which all of the zinc in the composition is in the active binder.
